(19) United States
(12) Patent Application Publication
(10) Pub. No.: US 2022/0017650 A1
(43) Pub. Date: Jan. 20, 2022

(54) NOVEL PROCESS FOR THE PURIFICATION OF SUGAMMADEX SODIUM

(71) Applicant: Biophore India Pharmaceuticals PVT. Ltd., Hyderabad, Telangana (IN)

(72) Inventors: Manik Reddy PULLAGURLA, Hyderabad, Telangana (IN); Bhaskar Reddy PITTA, Hyderabad, Telangana (IN); Suresh Babu NAMANA, Hyderabad, Telangana (IN); Maheswara Rao GOKADA, Hyderabad, Telangana (IN); Jagadeesh Babu RANGISETTY, Hyderabad, Telangana (IN)

(21) Appl. No.: 17/312,382

(22) PCT Filed: Dec. 11, 2019
(86) PCT No.: PCT/IB2019/060642

§ 371 Date: Jun. 9, 2021

(30) Foreign Application Priority Data
Dec. 11, 2018 (IN) 201841046764

(51) Int. Cl. C08B 37/16; C08L 5/16

(52) U.S. Cl. CPC C08B 37/0012; C08L 5/16

(57) ABSTRACT

The present invention relates to an improved process for the preparation of Sugammadex sodium (1). It further relates to a novel process for the purification of Sugammadex sodium (1) having impurity A less than 2% (w/w) and impurity E less than 0.1% (w/w)

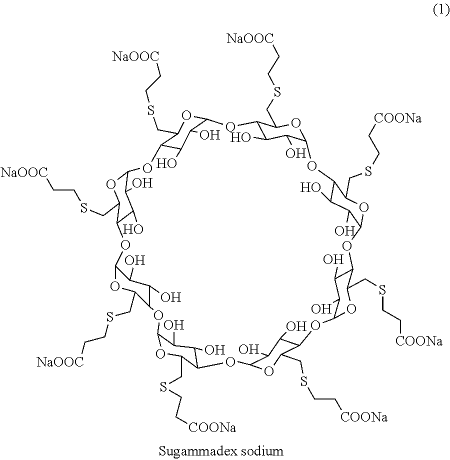

### CROSS REFERENCE

This application claims the priority from Indian Patent Application No. 201841046764 filed Indian Patent Office on Dec. 11, 2018.

### FIELD OF THE INVENTION

The present invention relates to an improved process for the preparation of Sugammadex or its pharmaceutically acceptable salt, preferably Sugammadex sodium (1). It further relates to a novel process for the purification of Sugammadex or its salts to provide Sugammadex sodium (1) with purity greater than 98.0%.

### BACKGROUND OF THE INVENTION

Sugammadex sodium (1) is an agent for reversal of neuromuscular blocking agents (NMBAs) rocuronium, vecuronium, pancuronium in general anesthesia. It is first selective relaxant binding agent (SRBA). Chemically it is 6^{A},6^{B},6^{C},6^{D},6^{E},6^{F},6^{G},6^{H}-Octakis-S-(2-carboxy ethyl) 6^{A},6^{B},6^{C},6^{D},6^{E},6^{F},6^{G},6^{H}-γ-cyclodextrin sodium salt (1:8). It is marketed by Merck under the trade name Bridion and was approved by the FDA in Dec. 15, 2015. Sugammadex was approved for use in the European Union on Jul. 29, 2008.

The following patents and applications describe the synthesis of Sugammadex sodium (1). U.S. Pat. No. 6,670,340 patent discloses preparation of Sugammadex sodium, by treating 6-per-deoxy-6-per-iodo-γ-Cyclodextrin with Sodium hydride and 3-Mercaptopropionic acid to form Sugammadex sodium. The reported process involves the preparation of Vilsmeier Hack reagent by the reaction of triphenylphosphine and iodine in dimethyl formamide, the obtained reagent is highly hygroscopic and difficult to handle in bulk scale. Another disadvantage of this method is removal of triphenylphosphine oxide (by-product) from the product, it requires more washings with dimethyl formamide that leads to loss of yield, tedious work up, lengthy process and not feasible on commercial scale.

U.S. Pat. No. 9,120,876 patent discloses the synthesis of Sugammadex sodium by reacting gamma cyclodextrin with phosphorous pentachloride to provide 6-perdeoxy-6-perchlorogammacyclodextrin. 6-perdeoxy-6-per-chloro gamma cyclodextrin is reacted with 3-mercaptopropionic acid in presence of alkali metal hydrides and an organic solvent to give 6-perdeoxy-6-per-(2-carboxyethyl) thio-γ-cyclodextrin sodium salt. Disadvantage of this process is the handling of phosphorous pentachloride in large scale synthesis and moreover highly exothermic while adding to reaction mixture, thus is not suitable at large scale, moreover workup procedure is tedious and lengthy.

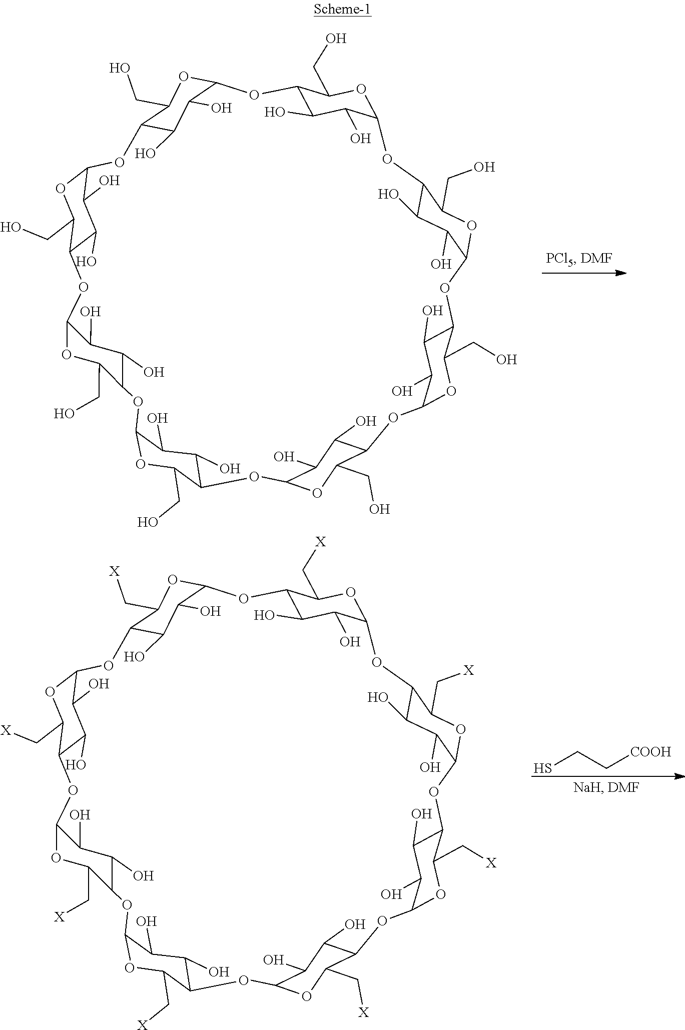

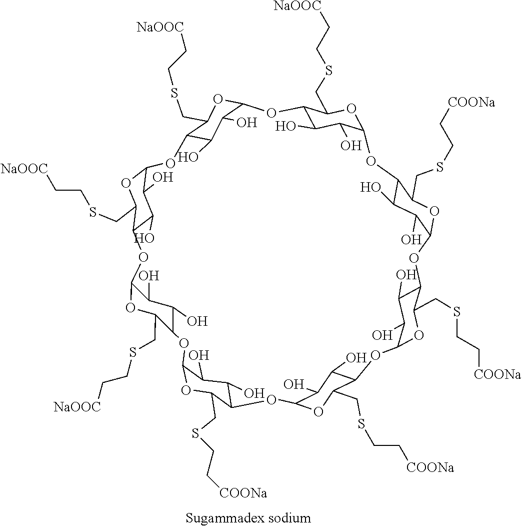

The purification techniques reported in the prior arts employ membrane dialysis, Sephadex G-25 column chromatography, macro porous resin, zeolites, molecular sieves, slurry with neutral aluminum oxide or basic aluminum oxide, use of which did not yield high purity Sugammadex. Also, these techniques are costly hence, economically not feasible and not convenient in large scale operations. Hence, the present inventors have reported a novel process for the preparation and a novel purification process of Sugammadex sodium (1) to obtain Sugammadex sodium (1) with greater than 98.0% purity by employing organic solvents followed by column purification, which makes it more feasible at industrial scale.

Major concerns in Sugammadex sodium is purity, removal of impurities in Sugammadex sodium is tricky and makes it unacceptable quality. Therefore, it is needed for efficient purification for high pure Sugammadex or its salts.

The present inventors hereby report a process for the preparation of pure Sugammadex or its salts by using simple purification process with high yield by using safe and commercially viable reagents under mild reaction conditions.

### OBJECTIVE OF THE INVENTION

Accordingly, one objective of the present invention is to provide an improved process for the preparation of Sugammadex free acid (5) or its pharmaceutically acceptable salt, preferably Sugammadex sodium (1).

Another objective of the invention is to provide novel process for the purification of Sugammadex free acid (5) or its pharmaceutically acceptable salt, preferably Sugammadex sodium (1).

Another objective of the invention is to provide Sugammadex sodium (1) with purity greater than 98.0% by HPLC (High performance liquid chromatography).

Yet, another objective of the invention is to provide Sugammadex free acid (5) or its sodium salt (1) with impurity A less than 3.0% (w/w), preferably less than 2.0% (w/w) and more preferably less than 1.0% (w/w) and impurity E less than 0.15% (w/w), preferably less than 0.1% (w/w).

### SUMMARY OF THE INVENTION

In one embodiment, the present invention provides an improved process for the preparation of Sugammadex sodium (1), which comprises:
a) reacting gamma cyclodextrin (4) with suitable halogenating agent in an organic solvent to provide 6-per-deoxy-6-perchloro gamma cyclodextrin (3);
b) reacting the 6-per-deox-6-perchloro gamma cyclodextrin intermediate (3) with mercaptopropionic acid or its salt form or its ester (2) in presence of base to yield Sugammadex sodium (1); and
c) purifying the Sugammadex sodium (1).

In another embodiment, the present invention provides novel process for the purification of Sugammadex or its salts, comprising:
A. providing a solution of Sugammadex or its salts in a suitable solvents or mixture thereof;
B. cooling the solution to a suitable temperature;
C. isolating Sugammadex or its salts by adding suitable protic solvent;
D. optionally, providing a solution of Sugammadex or its salts in a suitable biphasic solvent;
E. stirring the biphasic solution of Sugammadex or its salts at a suitable temperature;
F. isolating Sugammadex or its salts by cooling to a suitable temperature;
G. optionally, dissolving Sugammadex or its salts in water at a suitable temperature;
H. passing aqueous Sugammadex or its salts solution through flash column chromatography loaded with reverse phase silica gel; and
I. isolating pure Sugammadex or its salt by evaporating the pure fractions.

In another embodiment, the Sugammadex used in steps A, B and C may be pharmaceutically acceptable salt of Sugammadex, preferably Sugammadex sodium (1). Steps D, E and F involves use of Sugammadex free acid (5) or sodium salt, wherein Sugammadex sodium salt can be converted into free acid by treating with a suitable acid. Steps G, H and I involves use of Sugammadex sodium (1). Optionally, Sugammadex free acid may be converted to Sugammadex sodium (1) using a suitable base.

In another embodiment, Sugammadex free acid (5) or Sugammadex sodium (1) obtained after purification is having purity greater than 98.0% by HPLC.

In another embodiment, the present invention provides Sugammadex or its salts with impurity A less than 3.0% (w/w), preferably less than 2.0% (w/w) and more preferably less than 1.0% (w/w). Prior art methods do not disclose any simple methods for the removal of impurities.

### BRIEF DESCRIPTION OF DRAWINGS

FIG. 1: illustrates X-Ray powder diffraction (XPRD) pattern of Sugammadex sodium (1).

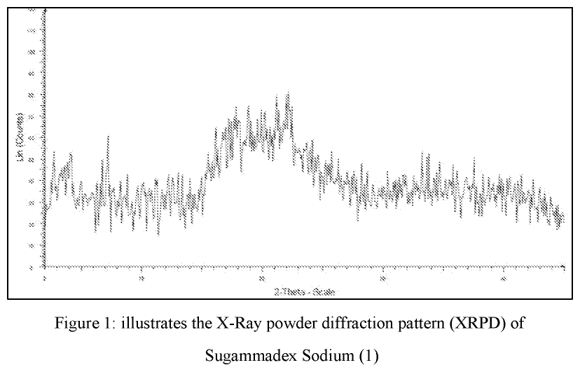

### DETAILED DESCRIPTION OF THE INVENTION

Accordingly, in one embodiment, the present invention provides, an improved process for the preparation of Sugammadex sodium (1) comprising:
a) reacting gamma cyclodextrin (4) with suitable halogenating agent in an organic solvent to provide 6-per-deoxy-6-perchloro gamma cyclodextrin (3);
b) reacting the 6-per-deox-6-perchloro gamma cyclodextrin intermediate (3) with mercaptopropionic acid (2) or its salt or its ester form in presence of base to yield Sugammadex sodium (1); and
c) purifying Sugammadex or its salts.

In another embodiment the present invention provides an improved process for the preparation of Sugammadex sodium (1), as shown in scheme 2.

Step a) involves reaction of gamma cyclodextrin (4) with suitable halogenating agent in a polar aprotic solvent to form 6-per-deoxy-6-chloro-gammacyclodextrin (3). The suitable halogenating agent may be selected from the group comprising of methane sulfonyl chloride, methane sulfonyl bromide, oxaloyl chloride, oxaloyl bromide or mixture thereof; preferably methane sulfonyl chloride was used in the present invention.

The organic solvent may be selected from the group comprising of polar aprotic solvents such as dimethylformamide, dimethyl sulfoxide, dimethylacetamide preferably dimethylformamide was used in the present invention.

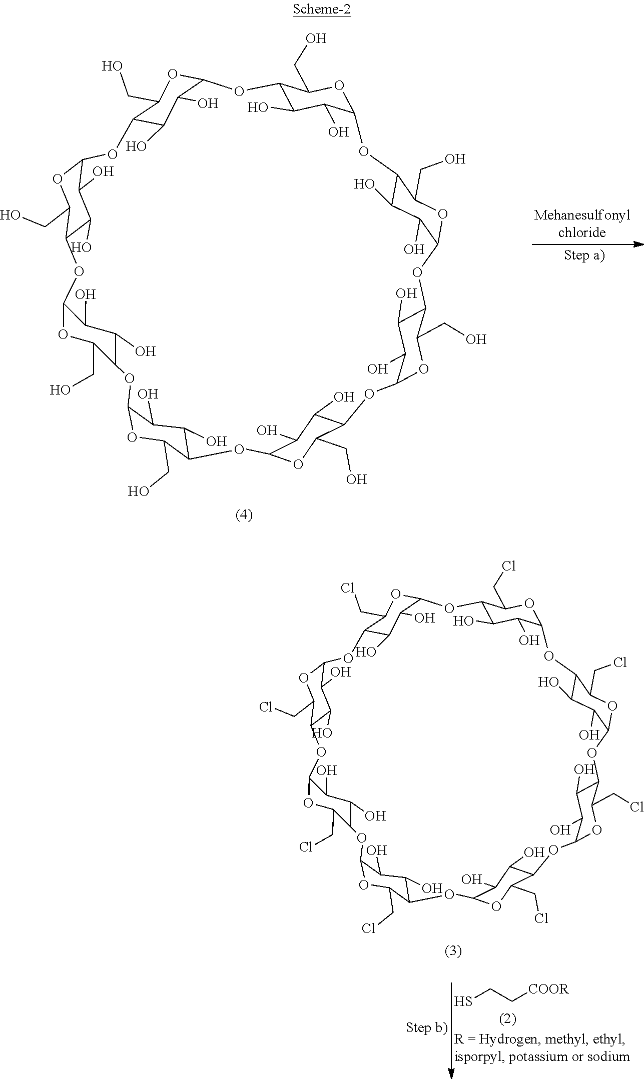

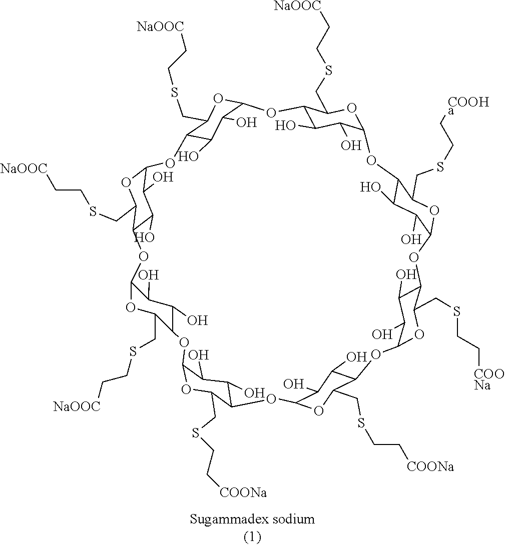

The amount of methane sulfonyl chloride used in step a) may be selected in the range of 8 to 20 molar ratio relative to gamma cyclodextrin, more preferably 15 to 20 molar ratios. The reaction temperature may be varied depending on the kind of polar aprotic solvent used. The said reaction in the present invention maybe carried out at a temperature about 40 to 70° C., more preferably 50-60° C.

Step b) proceeds with reacting 6-per-deoxy-6-chloro-gammacyclodextrin (3) with mercaptopropionic acid (2) or its salts or its ester compound in presence of base selected from alkali metal hydride or alkali metal alkoxide or alkali metal carbonates in a polar aprotic solvent to form Sugammadex or its salts. The mercaptopropionic acid (2) or its salts may be selected from sodium salt of mercaptopropionic acid or potassium salt of mercaptopropionic acid and mercaptopropionic acid (2) esters can be selected from methyl 3-mercaptopropionate, ethyl 3-mercaptopropionate, isopropyl 3-mercaptopropionate, preferably methyl 3-mercaptopropionate may be used.

The suitable alkali metal hydrides may be selected from the group comprising of sodium hydride and potassium hydride; alkali metal alkoxides may be selected from a group comprising of sodium methoxide, sodium ethoxide, potassium methoxide, sodium tertiary butoxide, potassium tertiary butoxide; alkali metal carbonates comprising of sodium carbonate, potassium carbonate, cesium carbonates or mixtures thereof. The polar aprotic solvent used may be selected from a group comprising of dimethylformamide, dimethyl sulfoxide, dimethylacetamide or mixtures thereof, preferably the reaction is carried out in dimethylformamide. The reaction temperature varies depending on the solvent used, but generally within a range of about 20-10° C., preferably from 70-80° C. The product may be isolated by adding a suitable polar solvent to the reaction mass. The suitable polar solvent includes methanol, ethanol, n-propanol, isopropanol, n-butanol, tertiary butanol, isoamyl alcohol, preferably isopropanol.

In another embodiment, the present invention provides novel process for the purification of Sugammadex or its salts. This novel purification process offers unexpected purity and is more efficient to remove impurities than the purification techniques known in the prior art.

The process of the purification Sugammadex or its salts comprises of the following steps:
A. providing a solution of Sugammadex or its salts in a suitable solvents or mixture thereof;
B. cooling the solution to suitable temperature;
C. isolating Sugammadex or its salts by adding suitable protic solvent;
D. optionally, providing a solution of Sugammadex free acid (5) in a suitable biphasic solvent;
E. stirring the biphasic solution of Sugammadex free acid (5) at suitable temperature;
F. isolating Sugammadex or its salts by cooling to a suitable temperature
G. optionally, dissolving Sugammadex or its salts in water at suitable temperature;
H. passing aqueous Sugammadex salt solution through flash column chromatography loaded with reverse phase silica gel; and
I. isolating pure Sugammadex or its salt by evaporating the pure fractions.

The step A proceeds with the preparation of Sugammadex or its salt solution in water and water miscible solvents. Mostly water was used to provide the solution and stirred for 20-30 minutes. The contents of step A so obtained may be cooled in step B to a temperature of 0-30° C., preferably 25-30° C. in some instances and 10-15° C. in other instances. Sugammadex or its salts can be isolated in step C by filtration or by adding suitable protic solvent to precipitate the solid. Steps A to C can be repeated for obtaining better purity.

In another embodiment, the Sugammadex used in steps A, B and C may be pharmaceutically acceptable salt of Sugammadex, preferably Sugammadex sodium (1). Optionally, Sugammadex sodium (1) may be converted to the Sugammadex free acid (5) using a suitable acid selected from sulphuric acid, acetic acid and hydrochloric acid. The step D involves preparing a solution of Sugammadex free acid (5) in a suitable biphasic solvent system and stirred continuously for 1-15 hrs, preferably 10-12 hrs, to reduce the stickiness of the solid and to aid easy filtration. The biphasic solvent system may be selected from a mixture of protic and aprotic solvent. The protic solvent which may be used is water. The aprotic solvents may be selected from organic solvents comprising from hydrocarbons, esters, or ethers. Hydrocarbon solvents may be selected from group of hexane, heptane, pentane, cyclohexane, toluene, xylene or mixture thereof. Ester solvents may be selected from group of methyl acetate, ethyl acetate, isopropyl acetate, isoamyl acetate, n-butyl acetate, isobutyl acetate or mixture thereof. Ether solvents may be selected from group of diethyl ether, di isopropyl ether, dibutyl ether, di-tertiary-butyl ether, methyl tert-butyl ether, ethyl tert butyl ether, dimethoxymethane, methyl t-butyl ether, glyme, diglyme, tetrahydrofuran, 2-methyltetrahydrofuran, 1,4-dioxane, dimethylfuran, anisole or mixture thereof. Preferably water and toulene mixture may be used in the present invention. The said reaction may be heated at 50-100° C., preferably at 70-75° C. The obtained reaction mass was isolated from the biphasic solvent mixture by cooling to 0-40° C., preferably 15-20° C.

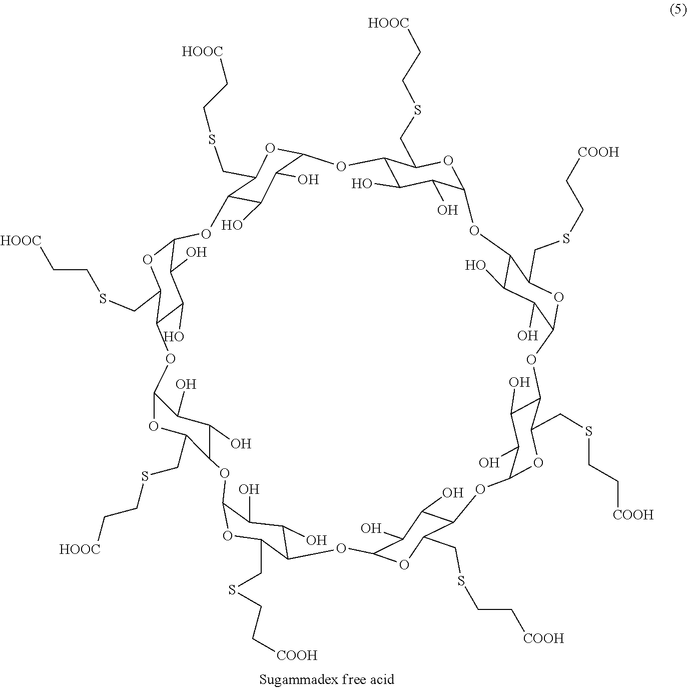 [SMILES: O=C(O)CCSCC1OC2OC3C(CSCCC(=O)O)OC(OC4C(CSCCC(=O)O)OC(OC5C(CSCCC(=O)O)OC(OC6C(CSCCC(=O)O)OC(OC7C(CSCCC(=O)O)OC(OC8C(CSCCC(=O)O)OC(OC9C(CSCCC(=O)O)OC(OC1C(O)C2O)C(O)C9O)C(O)C8O)C(O)C7O)C(O)C6O)C(O)C5O)C(O)C4O)C(O)C3O]

In another embodiment, Sugammadex free acid (5) obtained after biphasic purification in steps D, E and F is converted into sodium salt by treating with suitable base selected from the group comprising of sodium hydroxide, sodium methoxide, sodium tertiary butoxide or the like, preferably sodium hydroxide is used in the present invention.

The inventors have tried different processes for the purification of Sugammadex sodium (1) such as silica gel column chromatography, size exclusion chromatography and membrane dialysis to get high purity of Sugammadex sodium (1). Such purification techniques were not efficient for obtaining pure Sugammadex sodium (1), also was time consuming and not economically, and industrially viable. So, the present inventors found simple process for the purification of Sugammadex sodium (1) by using column chromatography.

The present invention provides purification of Sugammadex sodium (1) using column purification in steps G, H and I by using reverse phase silica gel. It can be carried out at normal temperature without applying any pressure, which is advantageous over preparative HPLC, since preparative HPLC is not industrially viable on large scale due to complex setups with larger volumes of solvents are also needed for the mobile phase.

In some embodiments, reverse phase silica gel used in the present invention can be selected from C8 and C18 bulk media with less than 200 microns. The mobile phase solvent may be selected from a mixture of protic solvents, acids and buffer. The protic solvents may be selected from a group comprising of water, methanol, ethanol or the like, the acid may be selected from trifluoro acetic acid, acetic acid, formic acid or the like and the buffer used may be selected from ammonium acetate, ammonium formate, ammonium carbonate or the like.

Preferably, mixture of water and trifluoro acetic acid and ammonium acetate was used in the present invention.

The step G proceeds with dissolving Sugammadex sodium (1) in water and loading to a column with reverse phase silica gel and eluting with mobile phase mixture of ammonium acetate, water and trifluoro acetic acid. The pure fraction collected were combined and adjusted pH to 8.0 to 8.5 using sodium hydroxide and distilled off under vacuum to isolate the pure Sugammadex sodium (1) having purity greater than 98% by HPLC.

In another embodiment, column purification is more effective in removing impurities. Specifically, controlling each impurity B, impurity C, impurity D and impurity E less than 0.1% (w/w) and any unspecified impurity can be controlled less than 0.1% (w/w).

In another embodiment, the Sugammadex sodium (1) obtained after biphasic purification in step D, E and F is having impurity A less than 3% (w/w) and preferably less than 2.0 (w/w) and more preferably less than 1.0% (w/w). Metal content is controlled according to ICH limits.

In another embodiment the below table-1 provides the impurity level before and after purification. The present purification methods are effective in controlling the process impurities and unknown impurities at less than 1.0% (w/w).

TABLE-1

| | | First | Second | Final |
|---|---|---|---|---|
| | Crude Sugammadex | Purification | Purification | Purification |
| | sodium or free acid | After Water/methanol | After Water/MTBE | After Column |
| | purity % (w/w) | purification % (w/w) | purification % (w/w) | purification % (w/w) |
| Impurity A | 10.0 | Less than 4.0 | Less than 1.0 | Less than 1.0 |
| Impurity B | 10-15 | 10-14 | 10-14 | Not detected |
| Impurity C | 5-7 | 5-6 | 5-6 | Not detected |
| Impurity D | 0.6-1.0 | 0.6-0.9 | 0.6-0.9 | Less than 0.03 |
| Impurity E | 0.6-1.0 | 0.4-1.0 | Less than 0.15 | Less than 0.15 |

From the above table-1, it is evident that combination of water and methanol followed by biphasic solvent purification water and methyl tertiary butyl ether (MTBE) is effective in reducing the impurity A and impurity E levels and column purification is effective in reducing the impurity B, impurity C and impurity D to not detectable levels. Sugammadex sodium purity can be further increase by repeating these purification methods. The said purification methods are effective in obtaining more purity and better yield.

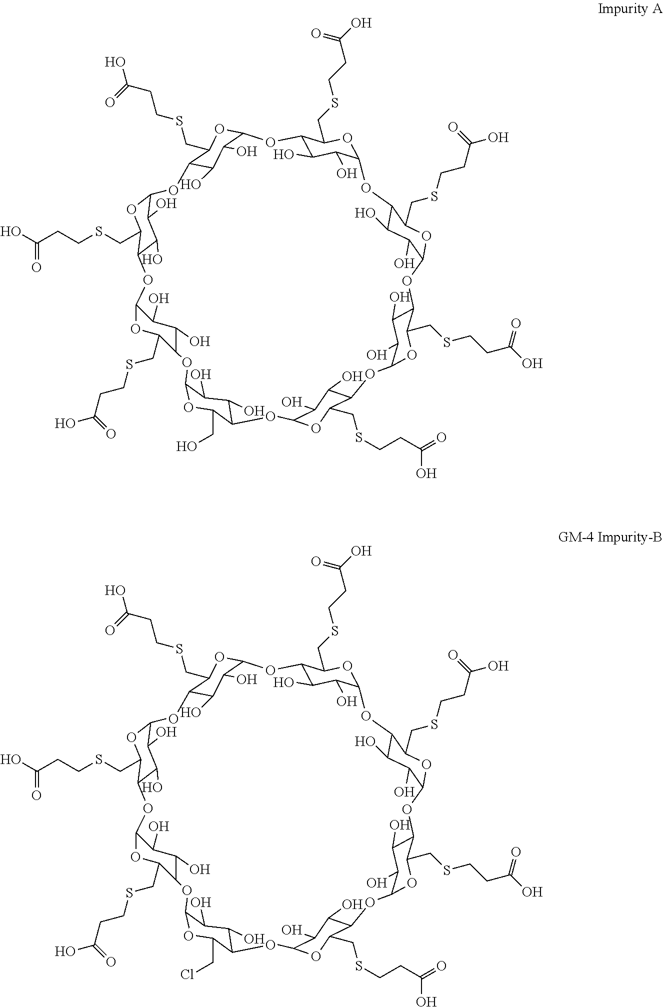 [SMILES: O=C(O)CCSCC1OC2OC3C(CSCCC(=O)O)OC(OC4C(CSCCC(=O)O)OC(OC5C(CSCCC(=O)O)OC(OC6C(CSCCC(=O)O)OC(OC7C(CSCCC(=O)O)OC(OC8C(CSCCC(=O)O)OC(OC9C(CO)OC(OC1C(O)C2O)C(O)C9O)C(O)C8O)C(O)C7O)C(O)C6O)C(O)C5O)C(O)C4O)C(O)C3O ; O=C(O)CCSCC1OC2OC3C(CSCCC(=O)O)OC(OC4C(CSCCC(=O)O)OC(OC5C(CSCCC(=O)O)OC(OC6C(CSCCC(=O)O)OC(OC7C(CSCCC(=O)O)OC(OC8C(CSCCC(=O)O)OC(OC9C(CCl)OC(OC1C(O)C2O)C(O)C9O)C(O)C8O)C(O)C7O)C(O)C6O)C(O)C5O)C(O)C4O)C(O)C3O]

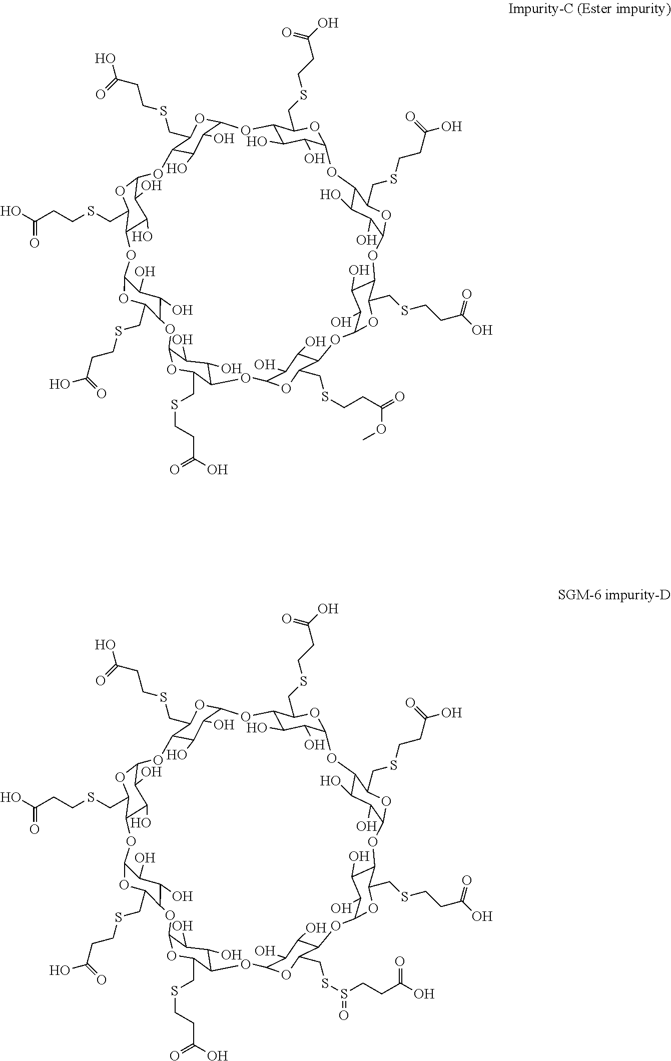 [SMILES: COC(=O)CCSCC1OC2OC3C(CSCCC(=O)O)OC(OC4C(CSCCC(=O)O)OC(OC5C(CSCCC(=O)O)OC(OC6C(CSCCC(=O)O)OC(OC7C(CSCCC(=O)O)OC(OC8C(CSCCC(=O)O)OC(OC9C(CSCCC(=O)O)OC(OC1C(O)C2O)C(O)C9O)C(O)C8O)C(O)C7O)C(O)C6O)C(O)C5O)C(O)C4O)C(O)C3O ; O=C(O)CCSCC1OC2OC3C(CSCCC(=O)O)OC(OC4C(CSCCC(=O)O)OC(OC5C(CSCCC(=O)O)OC(OC6C(CSS(=O)CCC(=O)O)OC(OC7C(CSCCC(=O)O)OC(OC8C(CSCCC(=O)O)OC(OC9C(CSCCC(=O)O)OC(OC1C(O)C2O)C(O)C9O)C(O)C8O)C(O)C7O)C(O)C6O)C(O)C5O)C(O)C4O)C(O)C3O]

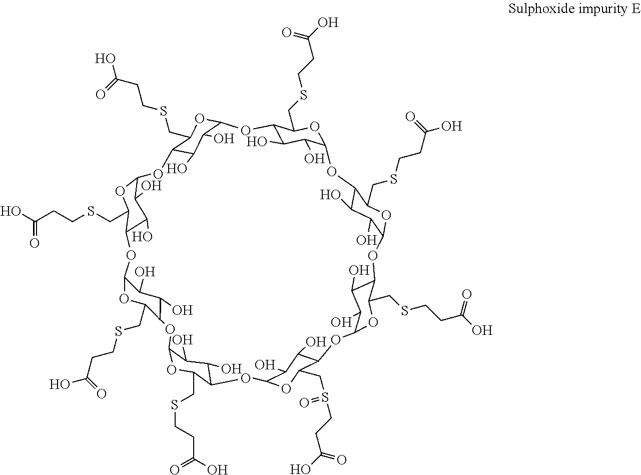 [SMILES: O=C(O)CCSCC1OC2OC3C(CSCCC(=O)O)OC(OC4C(CSCCC(=O)O)OC(OC5C(CSCCC(=O)O)OC(OC6C(CS(=O)CCC(=O)O)OC(OC7C(CSCCC(=O)O)OC(OC8C(CSCCC(=O)O)OC(OC9C(CSCCC(=O)O)OC(OC1C(O)C2O)C(O)C9O)C(O)C8O)C(O)C7O)C(O)C6O)C(O)C5O)C(O)C4O)C(O)C3O]

In some embodiment, the Sugammadex sodium (1) so obtained may be amorphous in nature.

The following examples further illustrate the present invention, but should not be construed in anyway, as to limit its scope

### EXAMPLES

### Example-1: Preparation of 6-per-deoxy-6-chloro-gammacyclodextrin (3)

100 g of gamma cyclodextrin (4) was added to 400 mL of dimethyl formamide and 263 g of methane sulfonyl chloride was added to the reaction mass at 25-30° C. The reaction mixture was heated at 55-60° C. for 2-4 days. The reaction mass was cooled and mixed with 1000 mL water at 5-10° C., stirred and filtered under vacuum. The solid obtained was washed with water and dried below 55° C. To the obtained solid, 400 mL of dimethyl formamide and 253.65 g of methane sulfonyl chloride dissolved in dimethylformamide were added and stirred for 12-15 hrs at 55-60° C. The solid obtained was treated with 100 mL of water and 300 mL of methanol at 25-30° C. and filtered under vacuum. The obtained solid was then washed with methanol and dried in an air tray dryer below 55° C. to obtain 6-per-deoxy-6-chloro-gammacyclodextrin (3), which was directly used in the next step. Yield: 90 g.

### Example-2: Preparation of Sugammadex Sodium (1)

99 g of 3-mercaptopropanoic acid (2) was dissolved in 720 mL of dimethyl formamide at 25-30° C. and 350 mL of 30% sodium methoxide solution was added to the reaction mass. Further, 90 g of the 6-per-deoxy-6-chloro-gammacyclodextrin (3) was added to the reaction mass and heated to 70-75° C. until completion of the reaction. The reaction mass was cooled to 25-30° C. and filtered under vacuum. The solid obtained was washed with 135 mL of methanol and dried under vacuum below 55° C. to obtain crude Sugammadex sodium (1). Yield: 200 g; Purity: 89%.

### Example-3: Preparation of Sugammadex Sodium (1)

16 g of anhydrous cesium carbonate was added to a solution of 9.0 g of 6-per-deoxy-6-chloro-gammacyclodextrin (3) and 6.65 mL of methyl 3-mercaptopropionate dissolved in dimethyl formamide at 25-30° C. The reaction mass was heated to 50° C. and the reaction mixture was added to 800 mL of water. The precipitate so obtained was filtered under vacuum and washed with water. The solid so obtained was treated with 10 mL of sodium hydroxide solution and stirred for 2-3 hrs at 25-30° C. The solution was passed through dialyzer for 6 hrs. The reaction mixture was then transferred in a flask, and the water was evaporated under vacuum to obtain Sugammadex sodium (1). Yield: 190 g

### Example-4: Alternate Process for the Preparation of Sugammadex Sodium (1)

90 g 6-per-deoxy-6-chloro-gammacyclodextrin (3) was added to 8 volumes of dimethylformamide and 9.0 g of methyl 3-mercaptopropionate was added to the reaction mass at 25-30° C. 200 g of cesium carbonate was added to the reaction mass and heated to 45-50° C. On completion of reaction, the reaction mass was cooled to 25-30° C., 8 volumes of water were added and filtered the solid. The solid was dissolved in 2 volumes water and 2 volumes sodium hydroxide at 25-30° C. The reaction mass was stirred for 2-3 hrs and concentrated under vacuum. The obtained solid was washed with 0.5 volume of methanol. Further, 1.25 volume of water was added to the solid and the reaction mass was cooled to 10-15° C. and then 2.5 volumes of acetic acid, 17 volumes of water added. Stirred the mixture for 15-20 minutes, filtered and washed the solid with water. The solid so obtained was dissolved in 2 volumes of water and heated to 60-65° C. The reaction mass was cooled to 25-30° C. and filtered the solid. The obtained solid was taken in a separate round bottom flask and added 2 volumes of water then cooled to 10-15° C. To this, sodium hydroxide solution was added at 10-15° C. to the reaction mass, filtered and washed with a mixture of 4 volumes of dimethyl sulfoxide and 1 volume of tetrahydrofuran. 2.5 volumes of acetic acid and methanol were added, stirred and filtered at 25-30° C. The wet mass was then treated with water, heated to 40-45° C., 10 volumes of methyl tertiary butyl ether added and cooled to 25-30° C. The solid formed was washed with methyl tertiary butyl ether and dried under vacuum. Finally, the solid so obtained was washed with methanol and converted to the sodium salt using 10 mL of sodium hydroxide solution and dried under vacuum below 50° C. to yield Sugammadex sodium (1). Yield: 30 g

### Example-5: Purification of Sugammadex Sodium (1)

200 g of crude Sugammadex sodium (1) was mixed in a mixture of 240 mL of water and 240 mL of methanol and 20 g of activated charcoal was added at 25-30° C. The reaction mixture was stirred for 20-30 minutes and filtered under vacuum. The solid so obtained was washed with water and filtered. 1700 mL of methanol was added to the obtained filtrate and the reaction mass was heated at 45-50° C. The reaction mass was cooled to 15-20° C., filtered and the solid was washed with methanol and dried under vacuum to obtain Sugammadex sodium (1). Yield: 100 g, Purity: 93%; Impurity A: less than 3% (w/w).

### Example-6: Purification of Sugammadex Free Acid (5)

100 g of Sugammadex sodium (1), was dissolved in 50 mL water and 300 mL of sulfuric acid was added to reaction mass at 25-30° C. 2000 mL of ethanol was then added to the reaction mass and filtered. The obtained wet solid was dissolved in 500 mL of water and heated at 65-70° C. The reaction mass was then cooled to 10-15° C. The solid formed was filtered and dried under vacuum to obtain Sugammadex free acid (5). The dried solid was further purified by using any below purification method mentioned.

### a) Purification of Sugammadex Free Acid (5)

100 g of Sugammadex free acid (5) was added to 1000 mL of toluene at 25-30° C. and stirred under nitrogen atmosphere. The resulting mixture was heated to 70-75° C., and 400 mL of water was added to the reaction mass. The reaction mass was then cooled to 25-30° C. and filtered under vacuum. The wet solid so obtained was dried under vacuum below 55° C. to obtain pure Sugammadex free acid (5). Yield: 80 g, Purity: >98.0%, Impurity A: less than 4%.

### b) Alternative Purification of Sugammadex Free Acid (5)

100 g of Sugammadex free acid (5) was added to 1000 mL of dimethoxymethane in at 25-30° C. and stirred under nitrogen atmosphere. The resulting mixture was heated to 70-75° C. and 400 mL of water was added to the reaction mass. The reaction mass was then cooled to 25-30° C. and filtered under vacuum. The wet solid so obtained was dried under vacuum below 55° C. to obtain pure Sugammadex free acid (5). Yield: 80 g, Purity: >98.0%

### c) Alternative Purification of Sugammadex Free Acid (5)

100 g of Sugammadex free acid (5) was charged to 1000 mL of n-butyl acetate in RBF at 25-30° C. and stirred under nitrogen atmosphere. The resulting mixture was heated to 70-75° C. and 400 mL of water was added to the reaction mass. The reaction mass was then cooled to 25-30° C. and filtered under vacuum. The wet solid so obtained was dried under vacuum below 55° C. to obtain pure Sugammadex free acid (5). Yield: 80 g, Purity: >98.0%; Impurity A: less than 3%.

### Example-7: Preparation of Sugammadex Sodium (1)

10 g Sugammadex free acid (5) was suspended in 10 mL of water and 20 mL of aqueous sodium hydroxide solution prepared by dissolving 1.6 g (8.0 equivalents) of sodium hydroxide in 20 mL of water and added to the reaction mass at 25-30° C. The obtained clear solution was stirred and then 700 mL of methanol was added and maintained at 25-30° C., then filtered the precipitated solid and dried under vacuum to obtain Sugammadex sodium (1). Yield: 10 g; Purity: 98.0%

### Example-8: Purification of Sugammadex Sodium (1) by Reverse Phase Column Chromatography

20 g of Sugammadex sodium was dissolved in 40 mL of water was loaded into the column packed with 60 g of reverse phase silica gel. This was eluted with a mobile phase comprising of 7.5 g of ammonium acetate dissolved in 1.0 L of water and 1.0 mL of trifluoro acetic acid for 5-6 hrs. The pure fraction collected were combined and adjusted pH with sodium hydroxide solution to 8.0 to 8.5 and distilled off under vacuum. The product Sugammadex sodium (1) was collected on the first few fractions. Purity: 98.4%.

### Example-9: One Pot Process for the Preparation of Sugammadex Sodium (1)

50 g of gamma cyclodextrin (4) was added to 200 mL of dimethyl formamide and 133 g of methane sulfonyl chloride was added to the reaction mass at 25-30° C. The reaction mixture was heated at 55-60° C. for 2-4 days. The reaction mass was cooled and mixed with 1000 mL water at 5-10° C., stirred and filtered under vacuum. The solid obtained was washed with water and dried below 55° C. To the obtained solid, 400 mL of dimethyl formamide and 253.65 g of methane sulfonyl chloride dissolved in dimethylformamide were added and stirred for 12-15 hrs at 55-60° C. The solid obtained was treated with 100 mL of water and 300 mL of methanol at 25-30° C. and filtered under vacuum. The obtained solid was then washed with methanol and dried in an air tray dryer below 55° C. to obtain 6-per-deoxy-6-chloro-gammacyclodextrin (3),

50 g of 3-mercaptopropanoic acid (2) was dissolved in 720 mL of dimethyl formamide at 25-30° C. and 350 mL of aqueous sodium methoxide solution was added. Further, above 6-per-deoxy-6-chloro-gammacyclodextrin (3) was added to the reaction mass and heated to 70-75° C. The reaction mass was cooled to 25-30° C. and filtered under vacuum. The solid obtained was washed with 75 mL of methanol and dried under vacuum below 55° C. to obtain crude Sugammadex sodium (1). The crude Sugammadex sodium (1) was mixed in a mixture of 100 mL of water and 100 mL of methanol and 10 g of activated charcoal was added at 25-30° C. The reaction mixture was stirred for 20-30 minutes and filtered under vacuum. The solid so obtained was washed with water and filtered. 700 mL of methanol was added to the obtained filtrate and the reaction mass was heated at 45-50° C. The reaction mass was cooled to 15-20° C., filtered and the solid was washed with methanol and dried under vacuum to obtain Sugammadex sodium (1). The Sugammadex sodium (1), was dissolved in 50 mL water and 150 mL of sulfuric acid was added to reaction mass at 25-30° C. 800 mL of ethanol was then added to the reaction mass and filtered. The obtained solid was dissolved in 500 mL of water and heated at 65-70° C. The reaction mass was then cooled to 10-15° C. The solid formed was filtered and dried under vacuum to obtain Sugammadex free acid (5). The Sugammadex free acid (5) was added to 600 mL of toluene at 25-30° C. and stirred under nitrogen atmosphere. The resulting mixture was heated to 70-75° C., and 400 mL of water was added to the reaction mass. The reaction mass was then cooled to 25-30° C. and filtered under vacuum. The wet solid so obtained was dried under vacuum below 55° C. to obtain pure Sugammadex free acid (5). Sugammadex free acid (5) was converted to Sugammadex sodium (1) by adding 20 mL of aqueous sodium hydroxide at 25-30° C. The obtained Sugammadex sodium was dissolved in water and passed through flash column chromatography using reverse phase silica gel and eluted with mixture of ammonium acetate, methanol and trifluoro acetic acid. The collected pure fractions were treated with aqueous sodium hydroxide solution to adjust pH to 8.0 to 8.5 and concentrated under vacuum to obtain pure Sugammadex sodium (1). Yield: 40 g, Purity: 99.07%; Impurity A: 0.8%; Impurity B and C: not detectable; Impurity D: 0.03%; Impurity E: 0.1%.

We claim:

1. A process for the preparation of Sugammadex sodium (1), comprising:
a) reacting gamma cyclodextrin (4) 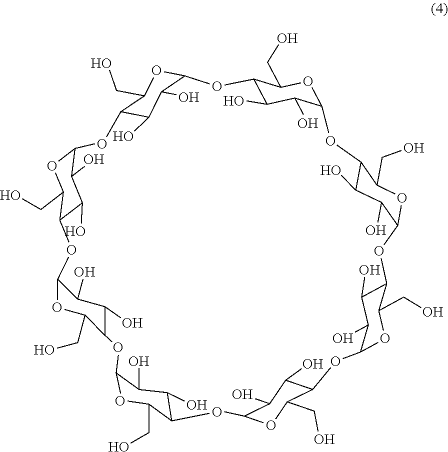 [SMILES: OCC1OC2OC3C(CO)OC(OC4C(CO)OC(OC5C(CO)OC(OC6C(CO)OC(OC7C(CO)OC(OC8C(CO)OC(OC9C(CO)OC(OC1C(O)C2O)C(O)C9O)C(O)C8O)C(O)C7O)C(O)C6O)C(O)C5O)C(O)C4O)C(O)C3O]
with suitable halogenating agent in a suitable organic solvent to provide 6-per-deoxy-6-perchloro gamma cyclodextrin (3); 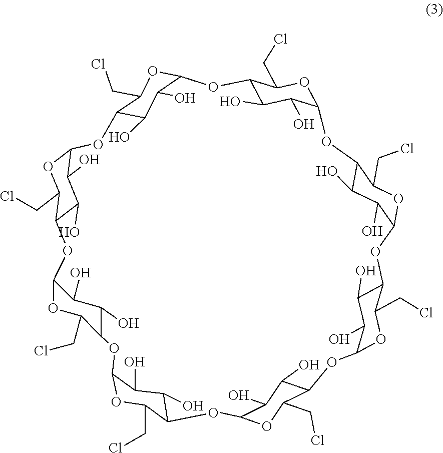 [SMILES: OC1C2OC(CCl)C(OC3OC(CCl)C(OC4OC(CCl)C(OC5OC(CCl)C(OC6OC(CCl)C(OC7OC(CCl)C(OC8OC(CCl)C(OC9OC(CCl)C(O2)C(O)C9O)C(O)C8O)C(O)C7O)C(O)C6O)C(O)C5O)C(O)C4O)C(O)C3O)C1O]
b) reacting the 6-per-deox-6-perchloro gamma cyclodextrin (3) with mercaptopropionic acid (2) or its salt or its ester 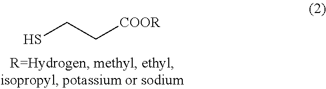
in presence of base to yield Sugammadex sodium (1); and
c) purifying Sugammadex sodium (1), wherein the purification comprises;
i. providing a solution of Sugammadex sodium (1) in a suitable solvents or mixture thereof;
ii. isolating Sugammadex sodium (1) by adding suitable solvent at a suitable temperature;
iii. optionally, converting Sugammadex sodium (1) to free acid (5) and dissolving in a suitable biphasic solvent;
iv. isolating Sugammadex free acid (5) by cooling to a suitable temperature;
v. optionally, converting Sugammadex free acid (5) to sodium salt (1) and dissolving in water;
vi. passing aqueous Sugammadex sodium (1) solution through flash column chromatography loaded with reverse phase silica gel; and
vii. isolating pure Sugammadex sodium (1) by evaporating the pure fractions.

2. The process as claimed in claim 1, wherein the suitable halogenating agent is selected from the group comprising of methane sulfonyl chloride, methane sulfonyl bromide, oxalyl chloride and oxalyl bromide.

3. The process as claimed in claim 1, wherein the salts and esters of mercaptopropionic acid (2) is selected from a group comprising of sodium_mercaptopropionic acid, potassium mercaptopropionic acid, methyl-3-mercaptopropionate, ethyl-3-mercaptopropionate, isopropyl-3-mercaptopropionate.

4. The process as claimed in claim 1, wherein the suitable base is selected from a group comprising of sodium hydride, potassium hydride, sodium methoxide, sodium ethoxide, potassium methoxide, sodium tertiary butoxide, potassium tertiary butoxide; sodium carbonate, potassium carbonate and cesium carbonates.

5. A compound of Sugammadex sodium (1) having purity greater than 98.0% by HPLC and one or more of the following:
i. impurity A less than 2.0% (w/w); and
ii. impurity E less than 0.1% (w/w)

6. The process as claimed in claim 5, wherein the purification process of Sugammadex sodium (1), having purity greater than 98.0% 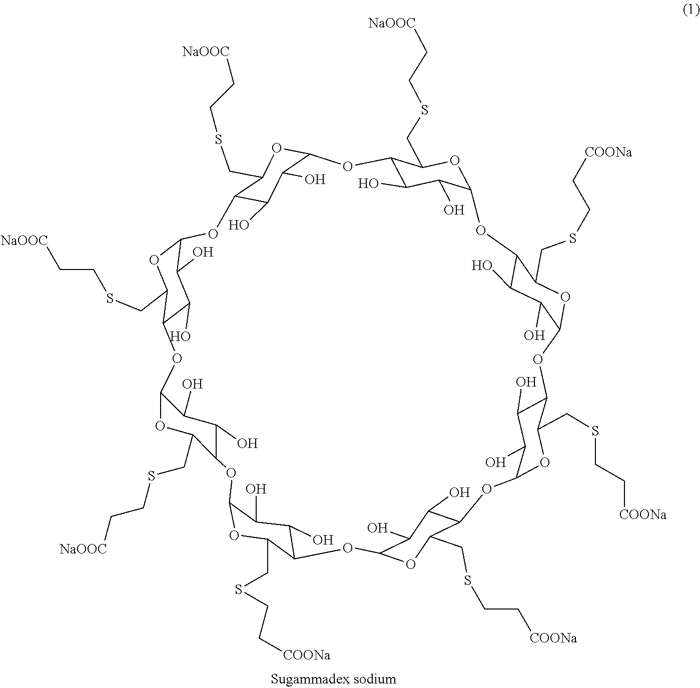
comprises of:
A. providing a solution of Sugammadex or its salts in a suitable solvents or mixture thereof;
B. cooling the solution to suitable temperature;
C. isolating Sugammadex or its salts by adding suitable protic solvent;
D. optionally, providing a solution of Sugammadex in a suitable biphasic solvent;
E. stirring the biphasic solution of Sugammadex at suitable temperature;
F. isolating Sugammadex or its salts by cooling to a suitable temperature;
G. optionally, dissolving Sugammadex or its salts in water at suitable temperature;
H. passing aqueous Sugammadex salt solution through flash column chromatography loaded with reverse phase silica gel; and
I. isolating pure Sugammadex or its salt by evaporating the pure fractions.

7. The process as claimed in claim 1 and claim 6, wherein the suitable solvent used herein is selected from water and suitable water miscible acid selected from the group comprising of hydrochloric acid, sulfuric acid, formic acid, acetic acid, propionic acid or mixture thereof.

8. The process as claimed in claim 1 and claim 6, wherein the suitable biphasic solvent system is selected from group comprising mixture of water and organic solvent. water immiscible solvent is selected from a group comprising of hexane, heptane, cyclohexane, toluene, methyl acetate, ethyl acetate, isopropyl acetate, diethyl ether, di isopropyl ether, dibutyl ether, di-tertiary-butyl ether, methyl tert-butyl ether, ethyl tertiary butyl ether, methyl t-butyl ether, tetrahydrofuran, 2-methyltetrahydrofuran, 1,4-dioxane, dimethylformamide, dimethylfuran, or mixture thereof.

9. A process for the purification of Sugammadex sodium (1) having purity greater than 98%, which comprises of:
a. providing a solution of Sugammadex sodium (1) in water at suitable temperature;
b. passing aqueous Sugammadex sodium (1) solution through flash column chromatography loaded with reverse phase silica gel; and
c. isolating pure Sugammadex or its salt by evaporating the pure fractions.

10. The process according to claim 9, wherein the suitable reverse phase silica gel used is selected from C8 and C18 silica gel.
